(19) United States
(12) Patent Application Publication
(10) Pub. No.: US 2013/0068711 A1
(43) Pub. Date: Mar. 21, 2013

(54) SURGICAL ASSEMBLY WITH VARIABLE POSITIONING ARM

(71) Applicants: Kelli N. Sebastian, Arlington, TN (US); John Stewart Young, Olive Branch, MS (US); David A. Mire, Cordova, TN (US)

(72) Inventors: Kelli N. Sebastian, Arlington, TN (US); John Stewart Young, Olive Branch, MS (US); David A. Mire, Cordova, TN (US)

(73) Assignee: WARSAW ORTHOPEDIC, INC., Warsaw, IN (US)

(21) Appl. No.: 13/238,087

(22) Filed: Sep. 21, 2011

(51) Int. Cl. A61B 19/00

(52) U.S. Cl. 211/85.13

(57) ABSTRACT

A surgical assembly includes an actuator. A linking member is connected to and extending from the actuator. The linking member includes a series of at least two interconnected inner links that are relatively movable such that the linking member is disposable between a flexible configuration and a rigid configuration. An arm extends from the actuator and includes a series of relatively moveable outer links that define an axial bore configured for disposal of the linking member. Each outer link defines a first mating surface and a second mating surface disposed in a configuration such that the actuator is engageable to move the linking member from the flexible configuration to the rigid configuration to selectively fix the arm in a selected orientation. Methods of use are disclosed.

### TECHNICAL FIELD

The present disclosure generally relates to medical devices, systems and methods employed during surgical applications, and more particularly, to a surgical assembly that includes a series of connected links configured to support a surgical instrument.

### BACKGROUND

Invasive surgical procedures such as open surgery for pathologies located within the body can cause significant trauma to intervening tissues. These procedures often require that the skin, tissue and/or vessels surrounding a surgical site be cut, removed, and/or repositioned so that a surgeon can access the site within the body. This trauma to the body may result in damage and scarring, as well as infection and long recovery.

Minimally invasive surgical procedures, including percutaneous techniques, are known that have attempted to overcome the above drawbacks of open surgery. Minimally invasive surgical procedures minimize disruption and trauma to the body to reduce recovery time and post-operative pain. For example, minimally invasive surgical techniques are employed for spinal and neurosurgical applications to access surgical sites within the body adjacent vital intervening tissues, in an effort to avoid damaging such vital tissues. Surgical retractors, mounting assemblies and other instruments are used in such minimally invasive procedures to increase the workspace of the minimally invasive surgical incision and adjacent areas used to access a surgical site. This disclosure describes an improvement over these prior art technologies.

### SUMMARY OF THE INVENTION

Accordingly, a surgical assembly, system and related methods are provided for employment during surgical applications. It is contemplated that the surgical assembly includes a series of connected links configured to support a surgical instrument and is used, for example, with a minimally invasive surgical procedure.

In one particular embodiment, in accordance with the principles of the present disclosure, a surgical assembly is provided. The surgical assembly includes an actuator having a proximal end and a distal end. A linking member is connected to and extending from the distal end of the actuator. The linking member includes a series of at least two interconnected inner links that are relatively movable such that the linking member is disposable between a flexible configuration and a rigid configuration. An arm extends from the distal end of the actuator and includes a series of relatively moveable outer links that define an axial bore configured for disposal of the linking member. Each outer link defines a first mating surface and a second mating surface disposed in a configuration such that the actuator is engageable to move the linking member from the flexible configuration to the rigid configuration such that the linking member drives the first mating surface into engagement with the second mating surface of an adjacent link in the series to selectively fix the arm in a selected orientation.

In one embodiment, the surgical assembly includes an actuator having a proximal end and a distal end, and includes an actuating member. A linking member has a proximal end and a distal end. The proximal end of the linking member is connected to and extending from the distal end of the actuator. The linking member includes a series of at least two interconnected inner links that are relatively movable such that the linking member is disposable between a flexible configuration and a rigid configuration. An arm extends from the distal end of the actuator and including a series of relatively moveable outer links that define an axial bore configured for disposal of the linking member. The series of outer links include a proximal link and a distal link. The proximal link is in communication with the actuating member and the distal link is connected to the distal end of the linking member. Each outer link defines a first mating surface and a second mating surface disposed in a configuration such that the actuator is engageable to move the linking member from the flexible configuration to the rigid configuration such that the linking member drives the first mating surface into engagement with the second mating surface of an adjacent link in the series to selectively fix the arm in a selected orientation.

In one embodiment, the surgical assembly includes an actuator having a proximal end and a distal end, and includes an actuating member. A linking member has a proximal end and a distal end. The proximal end of the linking member is connected to and extending from the distal end of the actuator. The linking member includes a series of at least two interconnected inner links that are relatively movable such that the linking member is disposable between a flexible configuration and a rigid configuration. An arm extends from the distal end of the actuator and includes a series of relatively moveable outer links that define an axial bore configured for disposal of the linking member. The series of outer links include a proximal link and a distal link. The proximal link is in communication with the actuating member and the distal link is connected to the distal end of the linking member. Each outer link defines a first mating surface and a second mating surface disposed in a configuration such that the actuator is engageable to move the linking member from the flexible configuration to the rigid configuration such that the linking member drives the first mating surface into engagement with the second mating surface of an adjacent link in the series to selectively fix the arm in a selected orientation. A mounting member is rotatably connected to the distal end of the actuator and rotatable about an axis traverse to a longitudinal axis of the actuator. An instrument connecting member is rotatably connected to the distal link of the arm and rotatable about an axis traverse to a longitudinal axis of the distal link.

### BRIEF DESCRIPTION OF THE DRAWINGS

The present disclosure will become more readily apparent from the specific description accompanied by the following drawings, in which:

FIG. 1 is a perspective view of one particular embodiment of a surgical assembly in accordance with the principles of the present disclosure;

FIG. 2 is a cross section view of the surgical assembly shown in FIG. 1;

FIG. 3 is a perspective view of the surgical assembly shown in FIG. 1 including a handle;

FIG. 4 is a perspective view of an outer link of the surgical assembly shown in FIG. 1;

FIG. 5 is a side, cross section view of the outer link shown in FIG. 4;

FIG. 6 is a perspective view of the outer link shown in FIG. 4;

FIG. 7 is an enlarged, break away cross section view of outer links of the surgical assembly shown in FIG. 1;

FIG. 8 is a side view of the surgical assembly shown in FIG. 1 including a clamp assembly;

FIG. 9 is an enlarged, break away cross section view of the surgical assembly shown in FIG. 8;

FIGS. 10 a and b are enlarged perspective views of a distal end of the surgical assembly shown in FIG. 1 illustrating connection with a surgical instrument;

FIG. 11 is a break away perspective view of the surgical assembly shown in FIG. 1 attached to a surgical instrument; and

FIG. 12 is a break away perspective view of the inner and outer links of the surgical assembly shown in FIG. 1.

Like reference numerals indicate similar parts throughout the figures.

### DETAILED DESCRIPTION OF THE INVENTION

The exemplary embodiments of the surgical assembly, related systems and methods of use disclosed are discussed in terms of medical devices employed during surgical applications and more particularly, in terms of a surgical assembly that includes a series of connected links configured to support a surgical instrument, which is used, for example, with a minimally invasive surgical procedure. It is envisioned that the surgical assembly, systems and methods of use disclosed provide a reliable and user-friendly mount and positioning assembly with a connected geometry of links, which is low profile to a patient allowing a surgeon facile access to a surgical site.

It is envisioned that the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. It is contemplated that the present disclosure may be employed with other osteal and bone related applications, including those associated with diagnostics and therapeutics. It is further contemplated that the disclosed surgical assembly may be employed in a surgical treatment with a patient in a prone or supine position, and/or employ various surgical approaches to the spine, including anterior, posterior, posterior mid-line, lateral, postero-lateral, and/or antero-lateral approaches, and in other body regions. The present disclosure may be employed with procedures for treating the lumbar, cervical, thoracic and pelvic regions of a spinal column. The system and methods of the present disclosure may also be used on animals, bone models and other non-living substrates, such as for training, testing and demonstration.

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”. Also, as used in the specification and including the appended claims, the term “tissue” includes soft tissue, ligaments, tendons, cartilage and/or bone unless specifically referred to otherwise.

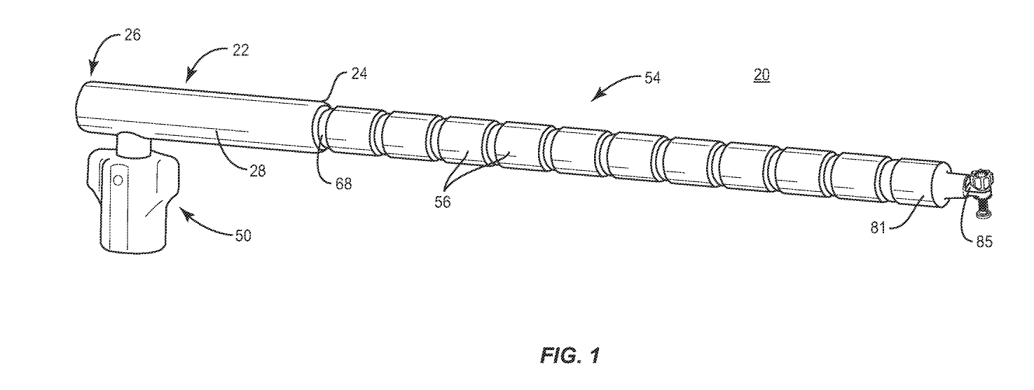

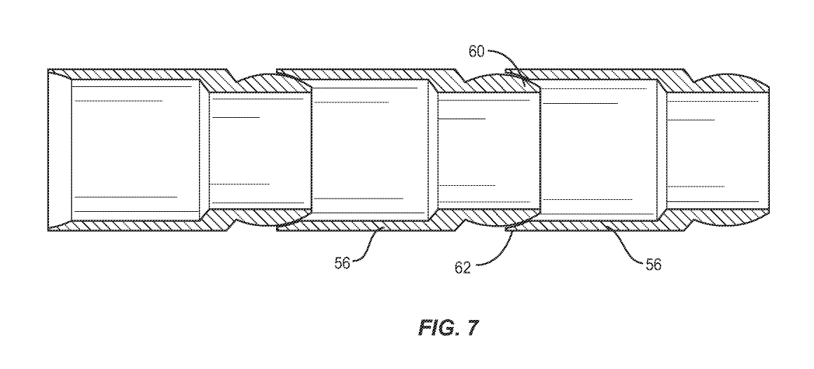

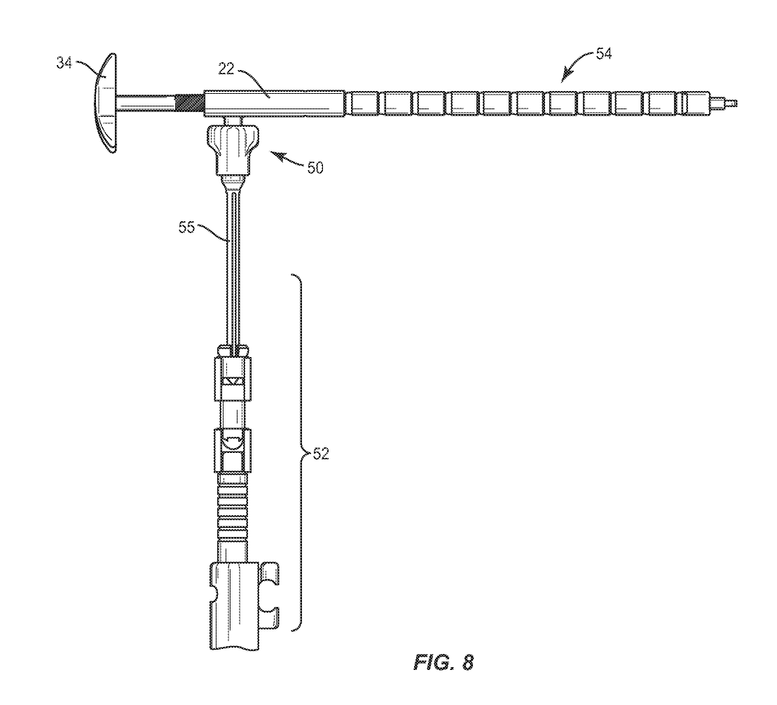

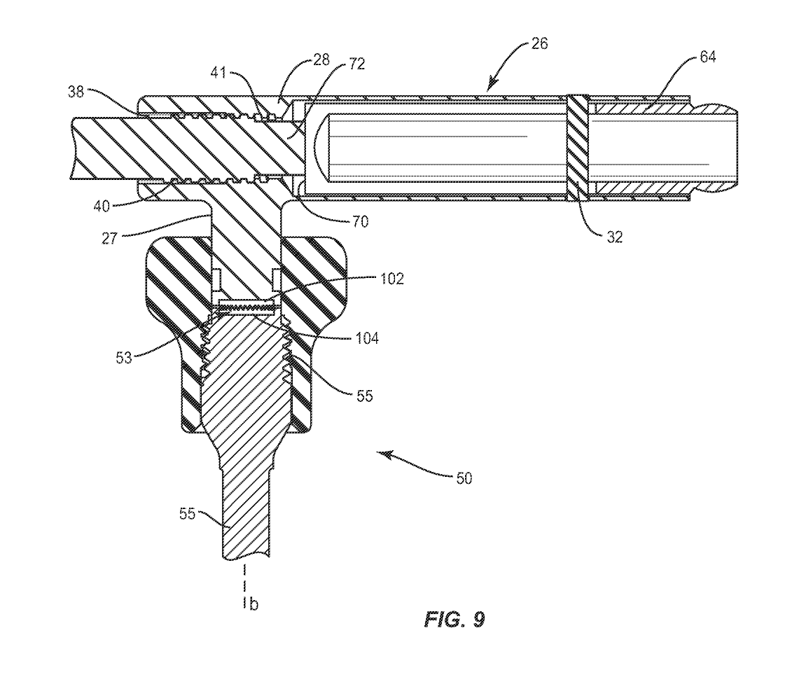

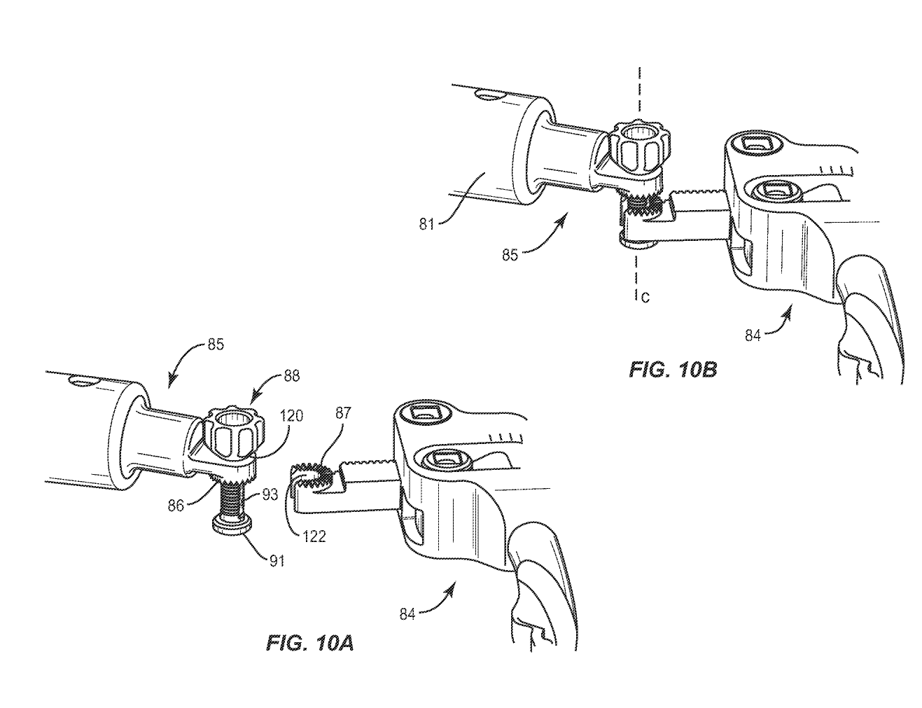

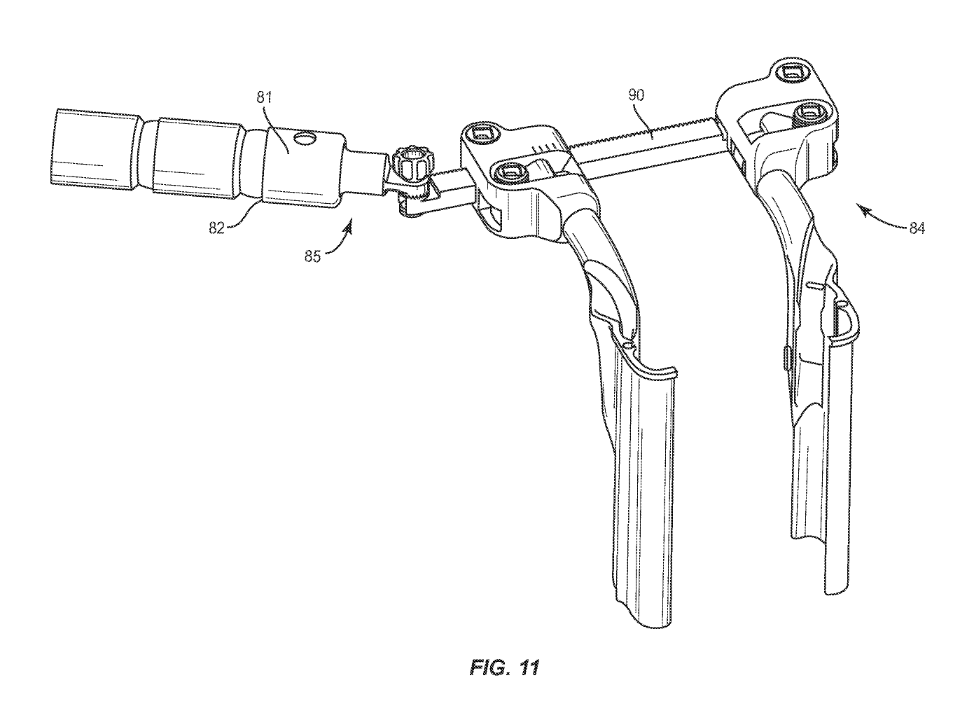

The following discussion includes a description of a surgical assembly, related components and exemplary methods of employing the surgical assembly in accordance with the principles of the present disclosure. Alternate embodiments are also disclosed. Reference will now be made in detail to the exemplary embodiments of the present disclosure, which are illustrated in the accompanying figures. Turning now to FIGS. 1-12, there is illustrated components of a surgical assembly 20 in accordance with the principles of the present disclosure.

The components of surgical assembly 20 and related systems are fabricated from materials suitable for medical applications, including metals, polymers, ceramics, biocompatible materials and/or their composites, depending on the particular application and/or preference of a medical practitioner. For example, the components of the surgical assembly, individually or collectively, can be fabricated from materials such as stainless steel, titanium, thermoplastics such as polyaryletherketone (PAEK) including polyetheretherketone (PEEK), polyetherketoneketone (PEKK) and polyetherketone (PEK), carbon-PEEK composites, PEEK-BaSO4 polymeric rubbers, biocompatible materials such as polymers including plastics, metals, ceramics and composites thereof, rigid polymers including polyphenylene, polyamide, polyimide, polyetherimide, polyethylene and epoxy. Various components of surgical assembly 20 may have material composites, including the above materials, to achieve various desired characteristics such as strength, rigidity, elasticity, compliance, biomechanical performance, durability and radiolucency or imaging preference. The components of surgical assembly 20, individually or collectively, may also be fabricated from a heterogeneous material such as a combination of two or more of the above-described materials. The components of surgical assembly 20 may be monolithically formed, integrally connected or include fastening elements and/or instruments, as described herein.

Referring to FIGS. 1-3, surgical assembly 20 includes an actuator 22, a linking member, such as, for example, an elongated chain link 30 and an arm 54. Surgical assembly 20 includes a mounting member 50 for mounting arm 54 in a fixed orientation relative to a surgical site, as will be described below. Surgical assembly 20 includes an instrument connecting member for connecting arm 54 to a surgical instrument, as will be described.

The actuator 22 includes a body 28 having a first end, such as, for example, a distal end 24 and a second end, such as, for example, a proximal end 26. It is contemplated that actuator 22 may be monolithically formed or integrally assembled. Actuator 22 includes a first member, such as, for example, a T-handle 34. T-handle 34 is connected to body 28 and extends into body 28 through a tubular cavity, such as, for example, a slot 38. T-handle 34 is manipulable for rotation thereof such that threads 40 engage internal threads 41 of body 28 to engage and axially translate arm 54 to tension chain link 30.

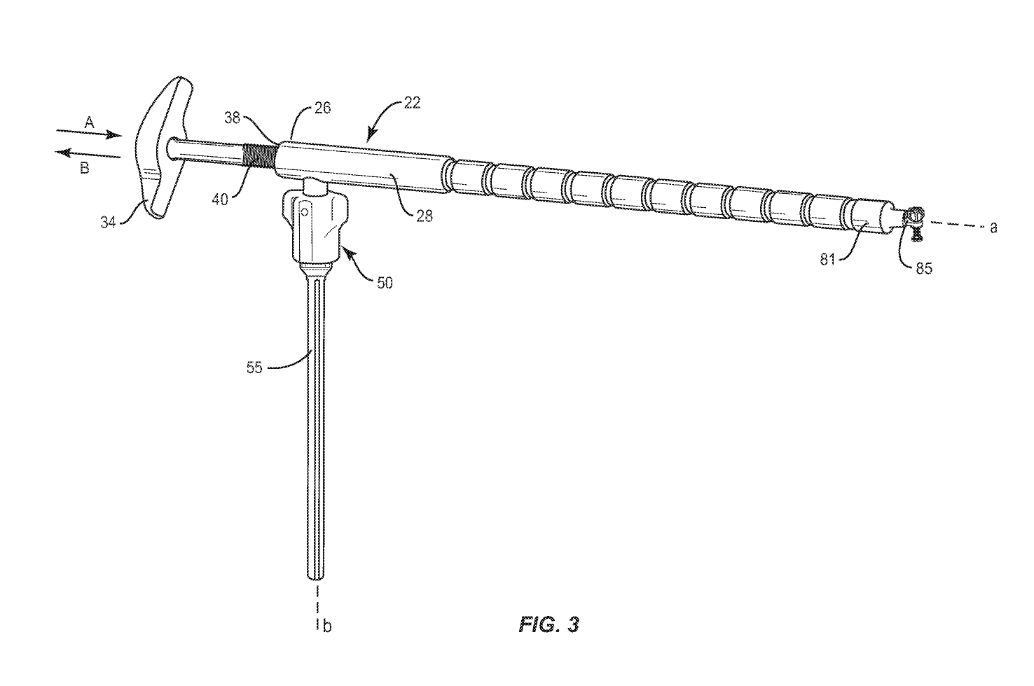

Upon clockwise rotation of T-handle 34, T-handle 34 is axially movable, in the direction shown by arrow A in FIG. 3, through slot 38 to drive the outer links of arm 54 distally and apply tension to chain link 30. This actuation, in the direction of arrow A, disposes the components of surgical assembly 20 in a fixed and/or rigid configuration. Upon counter clockwise rotation of T-handle 34, T-handle 34 is axially movable, in the direction shown by arrow B in FIG. 3, through slot 38 to draw the outer links of arm 54 proximally and release tension from chain link 30. This actuation, in the direction of arrow B, disposes the components of surgical assembly 20 in a flexible configuration. It is envisioned that T-handle 34 may be translated selective depths relative to body 28 to dispose the components of surgical assembly 20 in varying and/or selective degrees of tension to selectively dispose surgical assembly 20 between a flexible and fixed and/or rigid configuration.

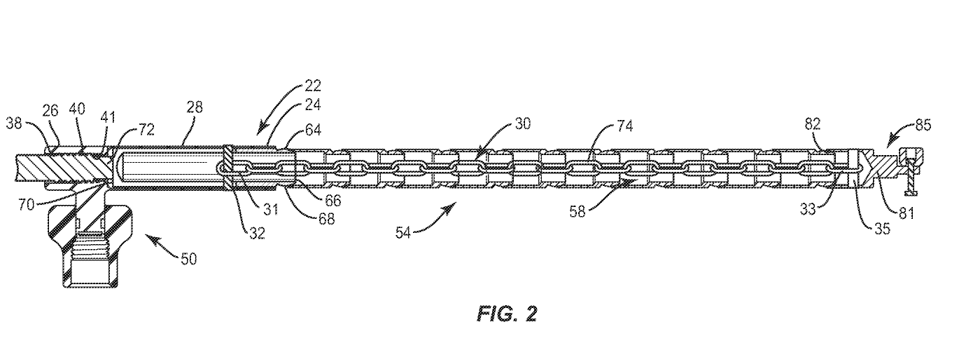

T-handle 34 includes a distal end 72, configured for connection with proximal link 64 of arm 54, as shown in FIG. 2, extending axially along slot 38 of body 28 to engage a proximal link 64 disposed at a proximal end thereof. As distal end 72 engages proximal link 64, the connected links of arm 54 are urged/forced distally in the direction of arrow A. Alternatively, proximal link 64 can be moved proximally in the direction of arrow B, via a release of tension that facilitates such movement, being drawn and/or pulled, according to the manipulation of T-handle 34. This engagement increases or decreases the tension of chain link 30 due to the connection of chain link 30 with body 28 and the distal end of arm 54. It is envisioned that actuator 22 may include an internal member that is threaded with the interior cavity of body 28 such that T-handle 34 is rotatable for axial translation of arm 54 and/or can be pushed/pulled for axial translation of arm 54. It is further envisioned that distal end 72 may be separate and engageable, attached with and/or formed with proximal link 64 to facilitate movement of arm 54. In one embodiment, the release of tension on arm 54 causes proximal movement of proximal link 64 due to a bias of arm 54 in the proximal direction.

Arm 54 extends from first end 24 and includes a series of relatively movable outer links 56. Outer links 56 collectively define an axial bore 58 extending along arm 54 and are configured for disposal of chain link 30. Each outer link 56 defines a portion of axial bore 58. In a linear configuration, arm 54 defines a longitudinal axis a. It is contemplated that arm 54 may be relatively movable to longitudinal axis a in one or a plurality of planes, directions and/or degrees of freedom, may be rotatable or twisted and/or axially movable.

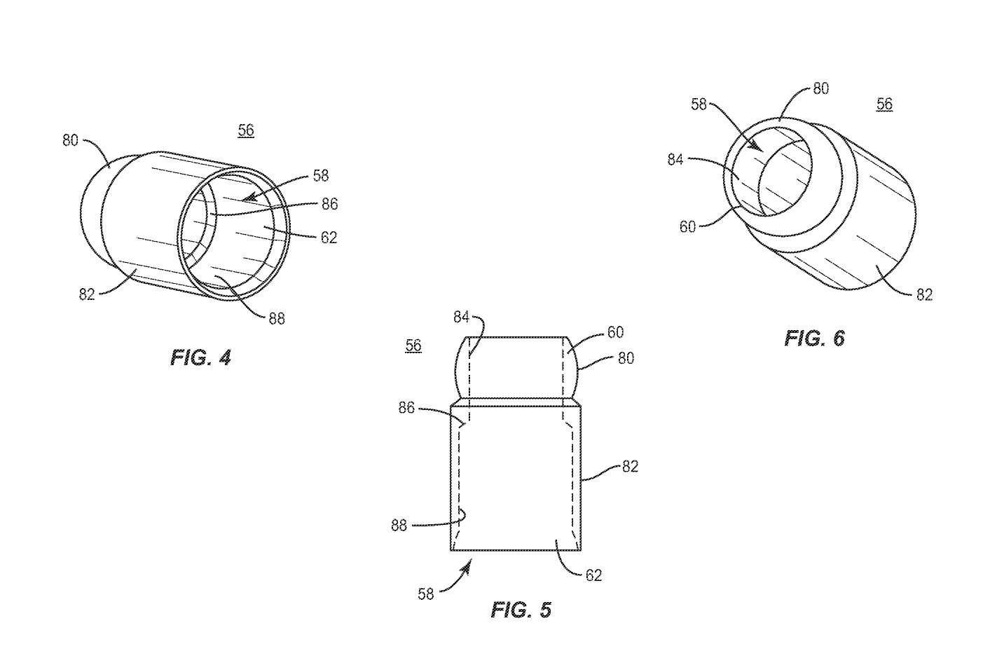

Each outer link 56, as shown in FIGS. 4-6, has a first end 80 having a spherical dome configuration and a second end 82 having a cylindrical configuration. Each outer link 56 defines a first cavity 84, a transition cavity 86 and a second cavity 88. Cavities 84/86/88 form a portion of axial bore 58 passing through each outer link 56 and a passageway for disposal of chain link 30. This configuration provides spherical dome articulation between outer links 56 and a conical socket for receiving the spherical dome of an adjacent outer link 56. Cavity 88 has a relatively larger dimension than cavity 84 to accommodate first end 60. Cavity 88 is defined by the conical socket, which has an arcuate surface of a particular outer link 56 to facilitate multi-axial motion relative to an adjacent outer link 56. It is contemplated that outer links 56 may be relatively movable in one or a plurality of planes, directions and/or degrees of freedom, may be rotatable or twisted and/or axially movable. It is further contemplated that one or all of outer links 56 may have alternate cross section configurations, such as, for example, oval, oblong, triangular, rectangular, square, polygonal, irregular, uniform, non-uniform, variable and/or tapered. It is further contemplated that one or all of outer links 56 may include fastening elements such as anchors, detents and/or openings for connection thereof. It is envisioned that outer links 56 may be relatively disposed and/or movable at alternate orientations from adjacent outer links 56, for example, perpendicular and/or other angular orientations such as acute or obtuse, co-axial, parallel and/or may be offset or staggered.

Each outer link 56 defines a first mating surface 60 and an opposing second mating surface 62. Mating surfaces 60, 62 are connected and configured to engage with a reciprocal mating surface of an adjacent outer link 56 in the series of links between a movable engagement for flexible articulation and orientation of arm 54, and a fixed engagement to fix arm 54 in a selected orientation for support and positioning of an instrument, as will be described. Axial bore 58 extends through mating surfaces 60,62 for disposal of chain link 30. Axial bore 58 has a circular cross section configuration. It is envisioned that axial bore 58 may have alternative cross section geometries, such as, for example, oval, oblong, triangular, rectangular, square, polygonal, irregular, uniform, non-uniform, variable and/or tapered.

In use, as the tension of chain link 30 is increased, the first mating surface 60 of each outer link 56 connects with and is configured to engage with second mating surface 62 of an adjacent outer link 56 in the series to selectively fix arm 54 in a desired orientation. First mating surface 60 engages an adjacent second mating surface 62 in a pressure and/or friction fit as outer links 56 are compressed together due to engagement of proximal link 64 with distal end 72, as described herein.

Proximal link 64 of arm 54 has a first end 66 including a mating surface 68, as shown in FIG. 2, connected to outer links 56 and configured to engage with first mating surface 62, similar to that described herein, and a second end 70 movably mounted with first end 24. This configuration selectively fixes an outer link 56 and an adjacent outer link 56 in a particular relative orientation such that arm 54 and surgical assembly 20 support an instrument in a position relative to a surgical site, according to the requirements of a particular application.

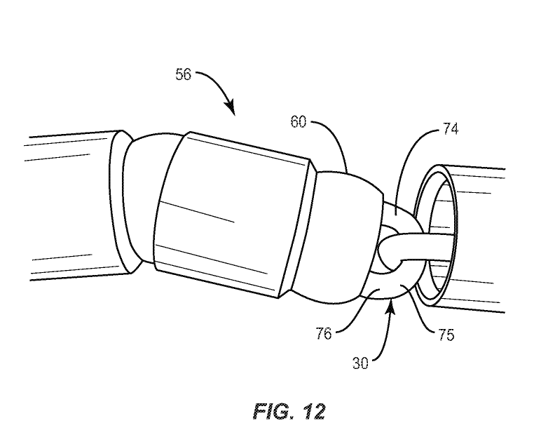

Chain link 30 includes a series of interconnected inner links 74, as shown in FIGS. 2 and 12, which are relatively movable such that chain link 30 is disposable between a flexible configuration, to facilitate articulation and orientation of arm 54 and a rigid configuration, to facilitate disposal of arm 54 in a fixed orientation for support and positioning of an instrument. Each inner link 74 has a toroid configuration with a solid, continuous link component 75 having an outer surface 76. The outer surface 76 of a particular inner link 74 is engageable with the outer surface 76 of an adjacent inner link 74 to facilitate fixation of chain link 30 in a rigid configuration. The continuous link components 75 are interconnected to form chain link 30. It is contemplated that all or only a portion of outer surface 75 may have alternate surface configurations, such as, for example, rough, undulating, porous, semi-porous, dimpled, polished and/or textured according to the requirements of a particular application and/or to enhance fixation of inner links 74 in a rigid configuration and/or as chain link is being tensioned.

Chain link 30 is attached to and extends from first end 24. Chain link 30 has a first end, such as, for example, a proximal end 31 attached to a pin 32 of actuator 22 and extends through axial bore 58 to a second end, such as, for example, a distal end 33 through outer links 56. Distal end 33 is attached to a distal end link 81 by pin 35. Chain link 30 transitions from the flexible configuration to the rigid configuration to facilitate support and positioning of a surgical instrument. Chain link 30 extends from pin 32 through proximal link 64 and into links 56. Second end 70 is engageable with the distal end 72 of T-handle 34 and proximal link 64 is movable with the series of links 56, as described above, to tension chain link 30. It is contemplated that proximal link 64 is axially movable and rotatable relative to first end 24 of body 28 to facilitate positioning of the instrument. It is further contemplated that proximal link 64 may be non-rotatable.

A mounting member 50 is connected to actuator 22 at second end 26, as shown in FIGS. 1-3 and 9. Actuator 22 includes a post 27 extending transversely from second end 26, along an axis b. Axis b is disposed relatively transverse to axis a. It is envisioned that axis b may be disposed relative to axis a at alternate orientations, for example, perpendicular and/or other angular orientations such as acute or obtuse, co-axial, parallel and/or may be offset or staggered.

Mounting member 50 is mounted with post 27 such that post 27 and arm 54 connected thereto are rotatable thereabout. This rotating configuration of arm 54 facilitates orientation and positioning of arm 54 and/or a surgical instrument connected to arm 54 relative to mounting member 50. It is contemplated that arm 54 may be rotated through an angle of 0-360 degrees about mounting member 50 and axis b.

Mounting member 50 permits actuator 22 and arm 54 to rotate about axis b of mounting member 50. Post 27 is attached to mounting member 50 via a splined mating geometry 53. Splined mating geometry 53 includes a spline 102 of post 27 having teeth that engage teeth of a spline 104 of an extender 55 of mounting member 50. In a non-locking configuration, the teeth of splines 102, 104 are relatively rotatable to facilitate relative rotation of post 27 and extender 55. Mounting member 50 includes internal threads 51 that mate with threads on extender 55 of mounting member 50. Upon desired positioning of arm 54, splines 102, 104 are actuated for disposal in a locked configuration. Mounting member 50 is rotated clockwise to axially translate mounting member 50 onto extender 55 and drive the teeth of splines 102, 104 into fixed engagement. This configuration locks position and orientation of arm 54 relative to mounting member 50, and thereby prevents further rotation of arm 54. Mounting member 50 is connected to an adjustable clamp assembly 52 configured for mounting with a fixture, such as, for example, a bed rail (not shown) or table.

In use, upon selective disposal of arm 54 in a selected orientation and position, the components of surgical assembly 20 are manipulated to dispose the components of surgical assembly 20 in a fixed and/or rigid configuration. The splined mating geometry 53 is locked, as discussed above, such that the position and orientation of arm 54 is fixed relative to mounting member 50. As such, further rotation of arm 54 relative to mounting member 50 is prevented.

T-handle 34 is rotated in a clockwise direction relative to body 28 such that threads 40 engage internal threads 41 of slot 38 to engage and axially translate proximal link 64 distally, in the direction shown by arrow A in FIG. 3, toward distal end link 81. Distal end 72 engages proximal link 64, which engages the series of outer links 56. Outer links 56 are driven distally such that outer links 56 are compressed distally. As such, distal end link 81 is driven distally such that pin 35 is pushed and/or driven by force distally to apply tension in the direction of arrow A to inner links 74.

As T-handle 34 is rotated, chain link 30 is selectively tensioned via engagement of outer links 56. As the tension of chain 30 is increased, first mating surface 60 interlocks with second mating surface 62 of an adjacent outer link 56 in the series to selectively fix links 56 in a selected orientation. This engagement moves arm 54 from the flexible configuration to the fixed and/or rigid configuration such that each outer link 56 is selectively fixed with an adjacent link 56 in a particular relative orientation such that arm 54 and surgical assembly 20 support an instrument in a position relative to a surgical site, according to the requirements of a particular application. It is envisioned that surgical assembly 20 may position an instrument in one or a plurality of orientations relative to a surgical site, and that arm 54 can be flexed in a plurality of orientations such as, multi-axial, linear, arcuate, helical and perpendicular and then fixed for orientation of an instrument. It is envisioned that the mating surfaces may be knurled, textured, form a friction or pressure fit engagement, notch and groove, single tooth and groove and/or include separate mechanical fixation such as a clamp. In the fixed and/or rigid configuration, it is envisioned that links 56 prevent undesired movement of arm 54.

Distal end link 81 has a mating surface 82 configured to engage and connect, similar to that described above, with first mating surface 60, and an instrument connecting member 85 for releasably engaging an instrument, such as, for example, a retractor assembly 84. Retractor assembly 84 is attached to instrument connecting member 85 via a splined mating geometry. Instrument connecting member 85 includes a spline 86 having teeth that engage teeth of a spline 87 of retractor assembly 84. The teeth of splines 86, 87 are relatively rotatable to facilitate rotation of retractor assembly 84 relative to arm 54.

Instrument connecting member 85 includes a threaded aperture 120 for disposal and threaded engagement with a threaded bolt 88. Aperture 120 is closed for retention of bolt 88 and includes spline 86 disposed on an outer surface thereof. Retractor assembly 84 includes a threaded open receiver 122 that is adaptable for mating with the spline surface of aperture 120 and bolt 88. Bolt 88 is threaded with aperture 120 and receiver 122, along an axis c, for mating arm 54 with retractor assembly 84 for disposal in a movable, non-locking configuration and a fixed, locked configuration. Axis c is disposed and movable relatively transverse to axis a and disposed and movable relatively parallel to axis b. It is envisioned that axis b may be disposed relative to axis a and/or axis b at alternate orientations, for example, perpendicular and/or other angular orientations such as acute or obtuse, co-axial, parallel and/or may be offset or staggered.

Upon selective positioning and orientation of retractor assembly 84 relative to instrument connecting member 85 and arm 54, splines 86, 87 are disposable in a locked configuration. A gripping surface, such as, for example, a knurled portion of bolt 88 is manipulated such that bolt 88 is threaded with aperture 120 and receiver 122. Bolt 88 includes opposing flat or planar surfaces 93 that align for engagement with corresponding flat surfaces of receiver 122 (not shown). Surfaces 93 engage the corresponding surfaces of receiver 122 to prevent rotation of bolt 88 during threading of the components to facilitate the travel and mating of the spline components. A stop 91 engages receiver 122 to provide an opposing force such that the teeth of splines 86, 87 are driven into fixed engagement. This configuration locks position and orientation of retractor assembly 84 relative to arm 54, and prevents further rotation of retractor assembly 84. Surgical assembly 20 and retractor assembly 84 are locked in position and orientation relative to a surgical site.

To disengage retractor assembly 84 from surgical assembly 20, screw 88 is rotated to separate splines 86, 87. Surgical assembly 20 can be repositioned and/or adjusted, to one or a plurality of orientations, by manipulating the splined mating geometries of mounting member 50 and/or retractor assembly 84 to a non-locked configuration. In the non-locked configuration, arm 54 can be rotated relative to mounting member 50 circumferentially about axis b and/or retractor assembly 84 can be rotated relative to arm 54 circumferentially about axis c. Surgical assembly 20 can be repositioned and/or adjusted, to one or a plurality of orientations, by releasing the tension of arm 54 and chain link 30, as described above.

In assembly, operation and use, the surgical system including surgical assembly 20 is employed, for example, with a minimally invasive surgical procedure for spinal and neurosurgical applications with a patient. For example, during minimally invasive spine surgery, a surgeon will make a small incision, typically less than one inch, in the skin of a patient's back over vertebrae to be treated. One or more dilators may be employed to gradually separate the muscles and create a portal through which the surgery may be performed.

Mounting member 50 of surgical assembly 20 is mounted to a bed rail, table mount or other fixture. Retractor assembly 84 is attached to arm 54, as described above. Arm 54 is rotated about axis b into position adjacent the surgical site over the small incision. The splined mating geometry of mounting member 50 is disposed in a locked configuration, as described, relative to axis b. Outer links 56 are manipulated into a desired position and orientation, as described, relative to the surgical site. Chain link 30 is tensioned such that arm 54 is fixed in the rigid configuration, as discussed.

Retractor assembly 84 is rotated about axis c into position adjacent the surgical site over the small incision. The splined mating geometry of retractor assembly 84 is disposed in a locked configuration, as described, relative to axis c. As facilitated by the configuration of surgical assembly 20, retractor assembly 84 may be positioned, repositioned and/or adjusted, to one or a plurality of orientations to perform a surgical procedure, according to the requirements of a particular application.

It is envisioned that the use of microsurgical and image guided technologies may be employed to access, view and repair spinal deterioration or damage, with the aid of surgical assembly 20. Upon completion of the procedure, the surgical instruments and assemblies are removed and the incision is closed. Alternatively, surgical assembly 20 may employed with an open spine surgery, which may involve making a long incision down the back, stripping large bands of muscle away from the spine and using retractor assembly 84 to retract, or pull the surrounding tissues and muscles to create a surgical site for treatment. It is envisioned that surgical assembly 20 may also be employed with mini-open surgery and percutaneous surgical implantation.

It is contemplated that a surgical procedure may employ other instruments that can be mounted with surgical assembly 20, such as, for example, nerve root retractors, tissue retractors, forceps, cutter, drills, scrapers, reamers, separators, rongeurs, taps, cauterization instruments, irrigation and/or aspiration instruments, illumination instruments and/or inserter instruments.

Surgical assembly 20 may be employed for performing spinal surgeries, such as, for example, discectomy, laminectomy, fusion, laminotomy, laminectomy, nerve root retraction, foramenotomy, facetectomy, decompression, spinal nucleus or disc replacement and bone graft and implantable prosthetics including plates, rods, and bone engaging fasteners.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A surgical assembly, comprising:
an actuator having a proximal end and a distal end;
a linking member connected to and extending from the distal end of the actuator, the linking member including a series of at least two interconnected inner links that are relatively movable such that the linking member is disposable between a flexible configuration and a rigid configuration; and
an arm extending from the distal end of the actuator and including a series of relatively moveable outer links that define an axial bore configured for disposal of the linking member, each outer link defining a first mating surface and a second mating surface disposed in a configuration such that the actuator is engageable to move the linking member from the flexible configuration to the rigid configuration such that the linking member drives the first mating surface into engagement with the second mating surface of an adjacent link in the series to selectively fix the arm in a selected orientation.

2. The surgical assembly of claim 1, further comprising a mounting member defining an axis and connected to the proximal end of the actuator,
wherein the axis of the mounting member is transverse to a longitudinal axis of the actuator, and
wherein the actuator is rotatable about the axis of the mounting member.

3. The surgical assembly of claim 2, wherein the mounting member comprises a first end having a locking member for securing the actuator in a fixed position and a second end connectable to a table mount.

4. The surgical assembly of claim 1, further comprising an instrument connecting member connected to a distal end outer link of the arm for releasably connecting to a surgical instrument, said connecting member having an axis of rotation through the connecting member,
wherein the axis of rotation is transverse to a longitudinal axis of the distal end outer link of the arm, and
wherein a connected surgical instrument is rotatable about the axis of rotation.

5. The surgical assembly of claim 4, wherein the instrument connecting member includes a splined configuration.

6. The surgical assembly of claim 4, wherein the distal end outer link of the arm is connected to a distal end inner link of the linking member.

7. The surgical assembly of claim 1, wherein the actuator includes a handle that is rotatable to apply a translational force onto a proximal end outer link to tension the inner links.

8. The surgical assembly of claim 7, wherein the actuator engages the proximal end outer link of the arm such that the arm translates in a first direction and draws the inner links in tension to tension the inner links.

9. The surgical assembly of claim 1, wherein the first mating surface has a convex configuration and the second mating surface has a concave configuration.

10. The surgical assembly of claim 1, wherein the axial bore of the arm extends through the first mating surface and the second mating surface.

11. The surgical assembly of claim 1, wherein each outer link is pivotable about a longitudinal axis of an adjacent outer link.

12. A surgical assembly, comprising:
an actuator having a proximal end and a distal end, and including an actuating member;
a linking member having a proximal end and a distal end, said proximal end of the linking member connected to and extending from the distal end of the actuator, the linking member including a series of at least two interconnected inner links that are relatively movable such that the linking member is disposable between a flexible configuration and a rigid configuration; and
an arm extending from the distal end of the actuator and including a series of relatively moveable outer links that define an axial bore configured for disposal of the linking member, said series of outer links including a proximal link and a distal link, said proximal link in communication with said actuating member and said distal link connected to the distal end of the linking member, each outer link defining a first mating surface and a second mating surface disposed in a configuration such that the actuator is engageable to move the linking member from the flexible configuration to the rigid configuration such that the linking member drives the first mating surface into engagement with the second mating surface of an adjacent link in the series to selectively fix the arm in a selected orientation.

13. The surgical assembly of claim 12, further comprising a mounting member defining an axis and connected to the proximal end of the actuator,
wherein the axis of the mounting member is transverse to a longitudinal axis of the actuator,
wherein the actuator is rotatable about the axis of the mounting member,
wherein the mounting member comprises a first end having a locking member for securing the actuator in a fixed position and a second end connectable to a table mount.

14. The surgical assembly of claim 12, further comprising an instrument connecting member connected to a distal end outer link of the arm for releasably connecting to a surgical instrument, said connecting member having an axis of rotation through the connecting member,
wherein the axis of rotation is transverse to a longitudinal axis of the distal end outer link of the arm, and
wherein a connected surgical instrument is rotatable about the axis of rotation.

15. The surgical assemble of claim 14, wherein the instrument connecting member includes a splined configuration.

16. The surgical assembly of claim 14, wherein the distal end outer link of the arm is connected to a distal end inner link of the linking member.

17. The surgical assembly of claim 1, wherein the actuator includes a handle that is rotatable to apply a translational force onto a proximal end outer link to tension the inner links.

18. The surgical assembly of claim 17, wherein the actuator engages the proximal end outer link of the arm such that the arm translates in a first direction and draws the inner links in tension to tension the inner links.

19. A surgical assembly, comprising:
an actuator having a proximal end and a distal end, and including a actuating member;
a linking member having a proximal end and a distal end, said proximal end of the linking member connected to and extending from the distal end of the actuator, the linking member including a series of at least two interconnected inner links that are relatively movable such that the linking member is disposable between a flexible configuration and a rigid configuration;
an arm extending from the distal end of the actuator and including a series of relatively moveable outer links that define an axial bore configured for disposal of the linking member, said series of outer links including a proximal link and a distal link, said proximal link in communication with said actuating member and said distal link connected to the distal end of the linking member, each outer link defining a first mating surface and a second mating surface disposed in a configuration such that the actuator is engageable to move the linking member from the flexible configuration to the rigid configuration such that the linking member drives the first mating surface into engagement with the second mating surface of an adjacent link in the series to selectively fix the arm in a selected orientation;
a mounting member rotatably connected to the distal end of the actuator rotatable about an axis traverse to a longitudinal axis of the actuator; and
an instrument connecting member rotatably connected to the distal link of the arm rotatable about an axis traverse to a longitudinal axis of the distal link.

20. The surgical assembly of claim 19, wherein the mounting member further comprises a first end having a locking member for securing the actuator in a fixed position and a second end connectable to a table mount, and
wherein the instrument connecting member includes a locking member for securing the instrument in a fixed position.
